(19) United States
(12) Patent Application Publication
(10) Pub. No.: US 2007/0072885 A1
(43) Pub. Date: Mar. 29, 2007

(54) BICYCLIC DERIVATIVES FOR THE TREATMENT OF ABNORMAL CELL GROWTH

(75) Inventors: Samit K. Bhattacharya, Groton, CT (US); Chandra A. Prakash, Gales Ferry, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/422,235

(22) Filed: Jun. 5, 2006

Related U.S. Application Data
(60) Provisional application No. 60/687,270, filed on Jun. 3, 2005.

(51) Int. Cl. A61K 31/517; C07D 403/02

(52) U.S. Cl. 514/266.21; 544/284

(57) ABSTRACT

The invention relates to substantially pure compounds of the formula 1 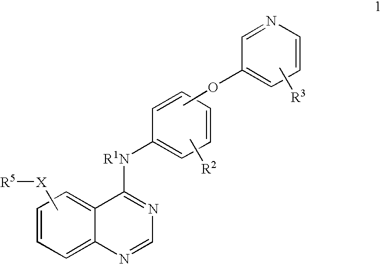
and to pharmaceutically acceptable salts, prodrugs and solvates thereof, wherein R¹, R², R³, R⁵, and X are as defined herein, and wherein the compound of formula 1 optionally further comprises a hydroxy substituent, an O-glucuronic acid, or an N−>O (N-oxide) moiety. The invention also relates to methods of treating abnormal cell growth in mammals by administering the compounds of formula 1 and to pharmaceutical compositions for treating such disorders that contain the compounds of formula 1. The invention also relates to methods of preparing the compounds of formula 1.

### CROSS-REFERENCE TO RELATED APPLICATION(S)

Reference is made to U.S. application Ser. No. 60/434,486, filed Dec. 18, 2002, and U.S. application Ser. No. 10/737,691 (Attorney Docket No. PC25074A), the disclosures of which are hereby incorporated by reference.

### BACKGROUND OF THE INVENTION

This invention relates to novel bicyclic derivatives that are useful in the treatment of abnormal cell growth, such as cancer, in mammals. This invention also relates to a method of using such compounds in the treatment of abnormal cell growth in mammals, especially humans, and to pharmaceutical compositions containing such compounds.

It is known that a cell may become cancerous by virtue of the transformation of a portion of its DNA into an oncogene (i.e., a gene which, on activation, leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Examples of receptor tyrosine kinases include c-erbB-2 (HER2), c-met, tie-2, PDGFr, FGFr, and VEGFR. It is known that such kinases are frequently aberrantly expressed in common human cancers such as breast cancer, gastrointestinal cancer such as colon, rectal or stomach cancer, leukemia, and ovarian, bronchial or pancreatic cancer. It is well known that ERBB2 protein tyrosine kinase erb B2 precursor (also known as c-erbB-2 protein precursor or kinase related transforming protein erbB2) is a protooncogene that encodes a membrane-bound receptor typrosine kinase of the epithelial growth factor receptor (EGFR) family. It is overexpressed in several types of cancer such as breast, ovarian, stomach, pancreus and colorectal cancers. ErbB2 has a possible role in tumor-cell proliferation, tumor invasion and tumor metastasis and drug resistance.

Accordingly, it has been recognized that inhibitors of receptor tyrosine kinases are useful as selective inhibitors of the growth of mammalian cancer cells. For example, erbstatin, a tyrosine kinase inhibitor, selectively attenuates the growth in athymic nude mice of a transplanted human mammary carcinoma which expresses epidermal growth factor receptor tyrosine kinase (EGFR) but is without effect on the growth of another carcinoma which does not express the EGF receptor. Thus, the compounds of the present invention, which are selective inhibitors of certain receptor tyrosine kinases, are useful in the treatment of abnormal cell growth, in particular cancer, in mammals. In addition to receptor tyrosine kinases, the compounds of the present invention can also display inhibitory activity against a variety of other non-receptor tyrosine kinases (eg: lck, src, abl) or serine/threonine kinases (e.g.: cyclin dependent kinases).

Various other compounds, such as styrene derivatives, have also been shown to possess tyrosine kinase inhibitory properties. More recently, five European patent publications, namely EP 0 566 226 A1 (published Oct. 20, 1993), EP 0 602 851 A1 (published Jun. 22, 1994), EP 0 635 507 A1 published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/340034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

### SUMMARY OF THE INVENTION

This invention relates to a substantially pure compound of the formula 1

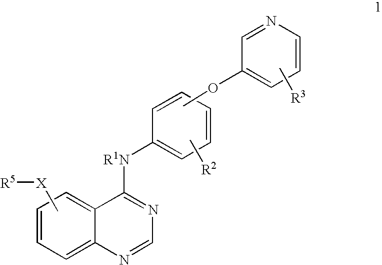

or a pharmaceutically acceptable salt, solvate or prodrug thereof, wherein:

X is selected from the group consisting of a bond —(CH═CH)— and —(CH₂—CH₂)— wherein any carbon atom in X may be optionally substituted with) R⁹;

R¹ is independently selected from the group consisting of hydrogen and —(C₁-C₆) alkyl;

R² is selected from the group consisting of hydrogen, hydroxyl, —(C₁-C₁₀) alkyl, —(C₁-C₆) alkoxy, and —(C₁-C₆) hydroxyalkyl group;

R³ is selected from the group consisting of hydrogen, —(C₁-C₆) alkyl, —(C₁-C₆) hydroxyalkyl, —CH₂OSO₂OH and —C(O)OR⁴, wherein R⁴ is selected from the group consisting of hydrogen and —(C₁-C₆) alkyl;

R⁵ is selected from the group consisting of —C(O)OH, —C(O)H—(CR⁶R⁷)ₘ—NR¹R⁵,

—CR⁶R⁷)ₘ—OH, —(CR⁶R⁷)ₘ—NR¹C(O)—CO₂H, —(CR⁶R⁷)ₘ—NR¹—C(O)—(CR⁶R⁷)ₘ—OH, and —(CR⁶R⁷)ₘ

—NR¹—(C(O))ₙ—(CR⁶R⁷)ₘ—O(C₁-C₆) alkyl, wherein m is an integer from 0 to 3 and wherein n is an integer from 1 to 2; each R⁶ and each R⁷ is independently selected from the group consisting of H and C₁-C₆ alkyl, and wherein R⁸ is selected from the group consisting of hydrogen, C₁-C₆ alkyl,

—C(O)—(C₁-C₆ alkyl) and —C(O)—(CR⁶CR⁷)ₘ—O(C₁-C₆ alkyl);

R⁹ is —S—(CR⁶R⁷)_{q}—CR¹(NR¹R⁸)—(CR⁸R⁷)ᵣ—C(O)OH, wherein q is an integer from 0 to 3 and wherein r is an integer fhom 0 to 2;

and wherein the compound of formula 1 is further optionally substituted by a hydroxy or an O-glucuronic acid substituent and wherein any nitrogen atom in the compound of formula 1 is optionally bound to an oxygen to form an N-oxide moiety (—N→O).

The invention also relates to a substantially pure compound of the formula 5

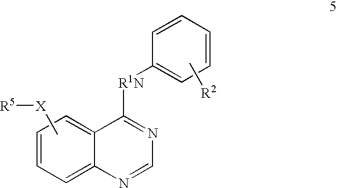

or a pharmaceutically acceptable salt, solvate or prodrug thereof, wherein:

X is selected from the group consisting of —(CH═CH)— and —(CH₂—CH₂)—;

R¹ is selected from the group consisting of hydrogen and —(C₁-C₆) alkyl;

R² is selected from the group consisting of hydrogen, hydroxy, —(C₃-C₁₀) alkyl, —(C₁-C₆) alkoxy, and —(C₁-C₆) hydroxyalkyl group;

R⁵ is selected from the group consisting of —C(O)OH, —C(O)H—(CR⁶R⁷)ₘ—NR¹R⁸, —(CR⁶R⁷)ₘ—OH, —(CR⁶R⁷)ₘ—NR¹C(O)—CO₂H, —(CR⁸R⁷)ₘ—NR¹—C(O)—(CR⁶R⁷)ₘ—OH, and —(CR⁶R⁷)ₘ—NR¹—(C(O))ₙ—(CR⁶R⁷)ₘ—O(C₁-C₆) alkyl, wherein m is an integer from 0 to 3 and wherein n is an integer from 1-2; each R⁶ and R⁷ is independently selected from the group consisting of H and C₁-C₆ alkyl, and wherein R⁸ is selected from the group consisting of H, C₁-C₆ alkyl, —C(O)—(C₁-C₆ alkyl) and —C(O)—(CR⁶CR⁷)ₘ—O(C₁-C₆ alkyl);

wherein the compound of formula 5 is further optionally substituted by a hydroxy or an O-glucuronic acid substituent; and wherein any nitrogen atom in the compound of formula 5 is optionally bound to an oxygen to form an N-oxide moiety (—N→O).

The invention also relates to a process for preparing a compound of the present invention by microbial biotransformation which comprises contacting a culture of a microorganism in a nutrient medium suitable for said microorganism with E-2-Methoxy-N-4(3-{4-[3-methyl-4-(6-methyl-pyridin-3-yloxy)-phenylamino]-quinazolin-6-yl}-allyl)-acetamide or a salt thereof and isolating the compound.

The invention also relates to a process for preparing a compound of the present invention, comprising the step of preparing the compound in vivo, and optionally isolating and purifying that compound.

The invention also relates to a process for preparing the compounds of the present invention, comprising the step of preparing the compound synthetically.

The invention also relates to a process for preparing E-N-(3-{4- [3-hydroxymethyl-4-(6-methyl-pyridin-3-yloxy)-phenylamino]-quinazolin-6-yl}-allyl)-2-methoxy-acetamide which comprises contacting a culture of the microorganism Streptomyces albulus in a nutrient medium suitable for said microorganism with the methanesulfonate salt of E-2-Methoxy-N-(3-{4-[3-methyl-4-(6methyl-pyridin-3-yloxy)-phenylamino]-quinazolin-6-yl}-allyl)-acetamide and isolating the E-N-(3-{4-[3-hydroxymethyl-4-(6-methyl-pyridin-3-yloxy)-phenylamino]-quinazolin-6-yl}-allyl)-2-methoxy-acetamide.

The invention also relates to a process for preparing E-N-(3-{4-[4-(6-hydroxymethyl-pyridin-3-yloxy)-3-methyl-phenylamino]-quinazolin-6-yl}-allyl)-2-methoxy-acetamide which comprises contacting a culture of the microorganism Streptomyces rimosus in a nutrient medium suitable for said microorganism with the methanesulfonate salt of E-2-Methoxy-N-(3-{4-[3-methyl-4-(6-methyl-pyridin-3-yloxy)-phenylamino]-quinazolin-6-yl}-allyl)-acetamide and isolating the E-N-(3-{4-[4-6-hydroxymethyl-pyridin-3-yloxy)-3-methyl-phenylamino]-quinazolin-6-yl-}-allyl)-2-methoxy-acetamide.

The invention also relates to a method for the treatment of abnormal cell growth (such as cancer) in a mammal comprising administering to said mammal an amount of a compound of the present invention that is effective in treating abnormal cell growth.

The invention also relates to a method for the treatment of abnormal cell growth in a mammal which comprises administering to said mammal an amount of a compound of the present invention that is effective in treating abnormal cell growth in combination with an anti-tumor agent selected tom the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

The present invention further relates to a pharmaceutical composition for the treatment of abnormal cell growth in a mammal comprising an amount of a compound of the present invention that is effective in treating abnormal cell growth, and a pharmaceutically acceptable carrier.

The present invention further relates to a method of determining if a patient has been administered E-2-Methoxy-N-(3-{4-[3-methyl-4-(6-methyl-pyridin-3-yloxy)-phenylamino]-quinazolin-6-yl}-allyl)-acetamide, the method comprising the step of determining if a plasma, urine, bile or fecal sample obtained from the patient shows the presence of the aforementioned compound of the present invention.

The present invention also relates to a kit for the treatment of abnormal cell growth comprising a) a pharmaceutical composition comprising a compound of the present invention and a pharmaceutically acceptable carrier, vehicle or diluent; and b) instructions describing a method of using the pharmaceutical composition for treating the abnormal cell growth.

### DETAILED DESCRIPTION OF THE INVENTION

This invention relates to a substantially pure compound of the formula 1

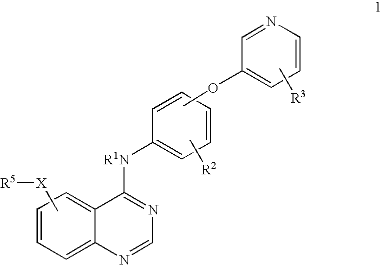

or a pharmaceutically acceptable salt, solvate or prodrug thereof, wherein:

X is selected from the group consisting of a bond. —(CH═CH)— and —(CH₂—CH₂)— wherein any carbon atom in X may be optionally substituted with R⁹;

R¹ is selected from the group consisting of hydrogen and —(C₁-C₆) alkyl;

R² is selected from the group consisting of hydrogen, hydroxyl, —(C₁-C₁₀) alkyl, —(C₁-C₆) alkoxy, and —(C₁-C₈) hydroxyalkyl group;

R³ is selected from the group consisting of hydrogen, —(C₁-C₆) alkyl —(C₁-C₆) hydroxyalkyl, —CH₂OSO₂OH and —C(O)OR⁴, wherein R⁴ is selected from the group consisting of hydrogen and —(C₁-C₆) alkyl;

R⁵ is selected from the group consisting of —C(O)OH, —C(O)H—(CR⁶R⁷)ₘNR¹R⁸, —(CR⁶R⁷)ₘ—OH, —(CR⁶R⁷)ₘ—NR¹C(O)—CO₂H, —(CR⁶R⁷)ₘ—NR¹—C(O)—(CR⁶R⁷)ₘ—OH, and —(CR⁶R⁷ₘ—NR¹—C(O))ₙ—(CR⁶R⁷)ₘ—O(C₁-C₆) alkyl, wherein m is an integer from 0 to 3 and wherein n is an integer from 1 to 2; each R⁸ and R⁷ is independently selected from the group consisting of H and C₁-C₆ alkyl, and wherein R⁶ is selected from the group consisting of H, C₁-C₆ alkyl, —C(O)—(C₁-C₆ alkyl) and —C(O)—(CR⁸CR⁷)ₘ—O(C₁-C₆ alkyl);

R⁹ is —S—(CR⁸R⁷)q—CR¹(NR¹R⁸)—(CR⁶R⁷)r—C(O)OH, wherein q is an integer from 0 to 3 and wherein r is an integer from 0 to 2; and wherein the compound of formula 1 is further optionally substituted by a hydroxy or an O-glucuronic acid substituent and wherein any nitrogen atom in the compound of formula 1 is optionally bound to an oxygen to form an N oxide moiety (—N→O).

In a preferred embodiment, the compound of the present invention is substantially pure. The substantially pure forms of the compound of the present invention can be obtained for example, through chemical synthesis, in vivo, or biotransformation, as set forth in detail below. In a preferred embodiment, the substantially pure forms of the compound of the present invention are obtained through chemical synthesis.

In one specific embodiment, the compound of formula 1, R¹ is H, R² is hydroxymethyl, R³ is methyl, and R⁵ is —CH₂NHC(O)CH₂OCH₃.

In another specific embodiment, R¹ is H, R² is methyl, R³ is hydroxymethyl, and R⁶ is —CH₂NHC(O)CH₂OCH₃.

In another specific embodiment, R¹ is H, R² is methyl, R³ is methyl and R⁵ is —C(O)OH.

In another specific embodiment, R¹ is H, R² is methyl, R³ is —COOH, and R⁶ —CH₂NHC(O)CH₂OCH₃.

In another specific embodiment, the compound of formula 1, R¹ is H, R² is methyl, R³ is methyl, and R⁵ is —CH₂NHC(O)CH₂OCH₃. In one embodiment the hydroxy moiety is a substituent within the bracketed portion of the molecule as shown below:

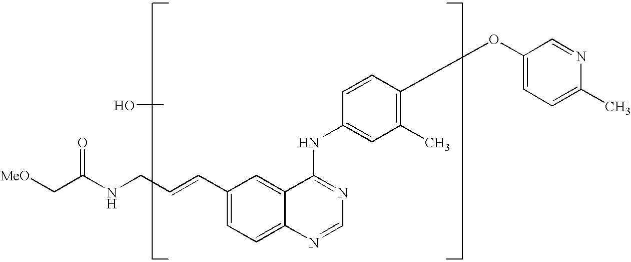

In one embodiment, the hydroxy moiety is a substituent within the bracketed part of the molecule as shown below:

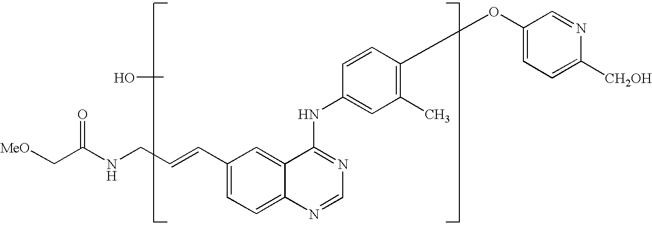

In another specific embodiment, R¹ is H, R² is hydroxymethyl, R³ is methyl, and R⁵ is —CH₂NHC(O)CH₂OH.

In another specific embodiment, the compound of formula 1 further comprises an —O-glucuronic acid substituent. In one embodiment the —O-glucuronic acid substituent is on the quinazoline ring; in one embodiment on the “phenyl” part of the phenylamino group; in one embodiment on the pyridine ring, and in the one embodiment on the acyclic chair attached to the phenyl group of the quinazoline ring.

In a further embodiment, the present invention provides a compound of formula 1 wherein one or more of the nitrogen atoms of a compound of formula 1 are bound to an oxygen to form an N-oxide moiety (i.e. —(N→O)).

In a preferred embodiment, the invention provides a compound of formula 1 including a moiety of formula 2: 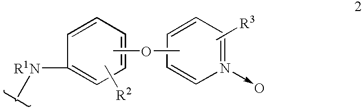
wherein R¹-R³, R⁵, and X are as defined above.

Alternatively, the invention also provides a compound of formula 1 including a moiety of formula 3: 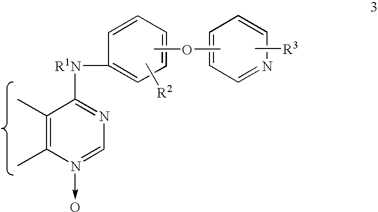
wherein R¹-R³, R⁵ and X are as define above.

Still further, the invention also provides a compound of formula 1 including a moiety of formula 4: 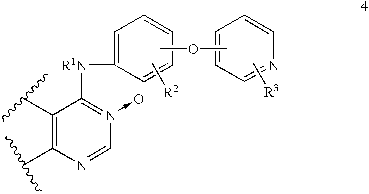
wherein R¹—R³, R⁵ and X are as defined above.

Still further the invention provides a substantially pure compound of the formula 5 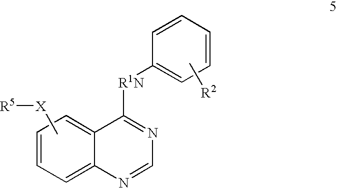
or a pharmaceutically acceptable salt, solvate or prodrug thereof, wherein:

X is selected from the group consisting of a —(CH═CH)— and —(CH₂—CH₂)—;

R¹ is selected from the group consisting of hydrogen and —(C₁-C₆) alkyl;

R² is selected from the group consisting of hydrogen, hydroxy, —(C₁-C₁₀) alkyl, —(C₁-C₆) alkoxy, and —(C₁-C₆) hydroxyalkyl group;

R⁵ is selected from the group consisting of —C(O)OH, —C(O)H—(CR⁶R⁷)ₘ—NR¹R⁸, —(CR⁶R⁷)ₘOH, —(CR⁶R⁷)ₘ—NR¹C(O)—CO₂H, —(CR⁶R⁷)ₘ—NR¹—C(O)—(CR⁵R⁷)ₘ—OH, and —(CR⁶R⁷)ₘ—NR¹—(C(O))ₙ—(CR⁶R⁷)ₘO(C₁-C₆) alkyl, wherein m is an integer from 0 to 3 and wherein n is an integer from 1-2; each R⁶ and R⁷ is independently selected from the group consisting of H and C₁-C₆ alkyl, and wherein R⁸ is selected from the group consisting of H, C₁-C₆ alkyl, —C(O)—(C₁-C₆ alkyl) and —C(O)—(CR⁶CR⁷)ₘ—O(CR₁-C₆ alkyl);

wherein the compound of formula 5 is further optionally substituted by a hydroxy or an O-glucuronic acid substituent; and wherein any nitrogen atom in the compound of formula 5 is optionally bound to an oxygen to form an N-oxide moiety (—N→O).

In a specific embodiment, R¹ is H, R² is OH, and R⁵ is —CH₂NHC(O)CH₂OCH₃.

Specific preferred compounds of the present invention include those selected from the group consisting of:
N-(3-{4-[3-hydroxymethyl-4-(6-methyl-pyridin-3-yloxy)phenylamino]-quinazolin-6-yl}-allyl)-2-methoxy-acetamide;
N-(3-{4-[4-(6-hydroxymethyl-pyridin-3-yloxy)-3-methyl-phenylamino]-quinazolin-6-yl}-allyl)-2-methoxy-acetamide;
3-{4-[3-Methyl-4-(6-methyl-pyridin-3-yloxy)-phenylamino]-quinazolin-6-yl}-acrylic acid;
5-{4-(6-[3-(2-Methoxy-acetylamino-propenyl]-quinazolin-4-ylamino}-2-methyl-phenoxy)-pyridine-2-carboxylic acid;
2-Hydroxy-N-(3-{4-[3-hydroxymethyl-4-(6-methyl-pyridin-3-yloxy)-phenylamino]-quinazolin-6-yl}-allyl)-acetamide.
N-{3-[4-(4-Hydroxy-3-methyl-phenylamino)-quinazolin-6-yl}-allyl]-2-methoxy-acetamide;

and the pharmaceutically acceptable salts, prodrugs, hydrates and solvates of the foregoing compounds.

Further preferred compounds of the present invention include those selected from the group consisting of:
2-Methoxy-N-(3-{4-[3-methyl-4-(6-methyl-1oxy-pyridin-3-yloxy)-phenylamino]-quinazolin-6-yl}-allyl)-acetamide;
2-Hydroxy-N-(3-{4-[3-methyl-4-(6-methyl-1-oxy-pyridin-3-yloxy)-phenylamino]-quinazolin-6-yl}allyl)-acetamide;
3-{4-[3-Methyl-4-(6-methyl-1-oxy-pyridin-3-yloxy)-phenylamino]-quinazolin-6-yl}-prop-2-en-1-ol;
N-(3-{4-[4-(6-Hydroxymethyl-1-oxy-pyridin-3-methyl-phenylamino]-quinazolin-6-yl}-allyl)-2-methoxy-acetamide;
N-[3-[4-(4-Hydroxy-3-methyl-phenylamino)-quinazolin-6-yl]-allyl)-2-methoxy-acetamide;
3-((-3-(2-methoxyacetamido)-1-(4-(4-(6-methylpyridin-3-yloxy)-3-methylphenylamino)quinazolin-6-yl)prop-1-enyl)sulfanyl)-2-aminopropanoic acid;
3-((-3-(2-methoxyacetamido)-1-(4-(4-(6-methylpyridin-3-yloxy)-3-methylphenylamino)quinazolin-6-yl)-prop-1-en-2-yl)sulfanyl)-2-aminopropanoic acid;
3-((-3-(2-methoxyacetamido)-1-(4-(4-6-methylpyridin-3-yloxy)-3-methylphenylamino)quinazolin-6-yl)prop-1-enyl)sulfanyl)-2-acetylpropanoic acid;
3-((-3-(2-methoxyacetamido)-1-(4-(4-(6-methylpyridin-3-yloxy)-3-methylphenylamino)quinazolin-6-yl)prop-1-en-2yl)-sulfanyl)-2-acetylpropanoic acid;
3-((-3-(2-methoxyacetamido-1-(4-4-(6-hydroxymethylpyridin-3-yloxy)-3-methylphenylamino)quinazolin-6-yl)prop-1-enyl)sulfanyl)-2-acetylpropanoic acid;
3-((-3-(2-methoxyacetamido)-1-(4-(4-(6-hydroxymethylpyridin-3-yloxy)-3-methylphenylamino)quinazolin-6-yl)prop-1-en-2-yl)sulfanyl)-2-acetylpropanoic acid; and
2-Amino-3-(1-hydroxy-3-{4-[3-methyl-4-(6-methyl-pyridin-3-yloxy)-phenylamino]-quinazolin-6-yl}-propylsulfanyl)-propionic acid;

and the pharmaceutically acceptable salts, prodrugs, hydrates and solvates of the foregoing compounds.

The compounds of the present invention can exist in both cis (Z) or trans (E) geometric isomeric forms. In one preferred embodiment of the present invention, the compounds of the present invention are E geometric isomers.

The compounds of the present invention may be used as analytical standards for in vitro or in vivo metabolism studies or as intermediates for the chemical synthesis or biosynthesis of new chemical entities. The metabolites may be isolated as solids or in solution. The compounds of the present invention can also be used to identify patients who have been administered E-2-Methoxy-N-(3-{4-[3methyl-4-(6-methyl-pyridin-3-yloxy)-phenylamino]-quinazolin-6-yl}-allyl)-acetamide or a pharmaceutically acceptable salt or prodrug thereof, or salt of a prodrug. To identify a patient that has been administered E-2-Methoxy-N-(3-{4-[3-methyl-4-(6-methyl-pyridin-3-yloxy)-phenylamino]-quinazolin-6-yl}-allyl)-acetamide or a pharmaceutically acceptable salt or prodrug thereof or salt of a prodrug, a serum, urine, fecal or bile sample is taken from the patient and the sample is analyzed for the presence of one or more compound of the present invention.

One method of analyzing the compounds of the present invention is by using chromatography and mass spectroscopy. Other analysis methods are well known to those skilled in the art. The presence of one or more compound of the present invention in a serum, urine, fecal or bile sample indicates that the patient has been administered E-2-Methoxy-N-(3-{4-[3-methyl-4-(6-methyl-pyridin-3-yloxy)-phenylamino]-quinazolin-6-yl}-ally)-acetamide or a pharmaceutically acceptable salt or prodrug thereof, or salt of a prodrug.

In the methods of treatment of the present invention, a compound of the present invention can be administered to a patient directly, such as in a tablet, or the compound can be administered by being produced in the patients body through metabolism. Moreover, the administration route and dosage of the compound that gives rise to a compound of the present invention by metabolism can be varied, as desired, to obtain desired in vivo concentration and rate of production of a compound of the present invention.

This invention also relates to a process for preparing the compound of the present invention by microbial biotransformation which comprises contacting a culture of a microorganism in a nutrient medium suitable for said microorganism with E-2-Methoxy-N-(3-{4-[3-methyl-4-(6-methyl-pyridin-3-yloxy)-phenylamino]-quinazolin-yl}-allyl-acetamide or a salt thereof and isolating the compound.

In one embodiment, the microorganism is an actinomycete, and in one embodiment a fungus.

This invention also relates to a process for preparing E-N-(3-{4-[3-hydroxymethyl-4-(6-methyl-pyridin-3-yloxy)-phenylamino]-quinazolin-6-yl}-allyl)-2-methoxy-acetamide which comprises: contacting a culture of the microorganism Streptomyces albulus in a nutrient medium suitable for said microorganism with the methanesulfonate salt of E-2Methoxy-N-(3-{4-[3-methyl-4-(6-methyl-pyridin-3-yloxy)-phenylamino]-quinazolin-6-yl}-allyl)-acetamide and isolating the E-N-(3-{4-[3-hydroxymethyl-4-(6-methyl-pyridin-3-yloxy)-phenylamino]-quinazolin-6-yl}-allyl)-2-methoxy-acetamide.

In one preferred embodiment the nutrient medium suitable for Streptomyces albulus is IOWA medium.

This invention also relates to a process for preparing E-N-(3-[4-[4-(6-hydroxymethyl-pyridin-3-yloxy)-3-methyl-phenylamino]-quinazolin-6-yl-allyl)-2-methoxy-acetamide which comprises contacting a culture of the microorganism Streptomyces rimosus in a nutrient medium suitable for said microorganism with the methanesulfonate salt of E-2-Methoxy-N-(3-{4-[3-methyl-4-(6-methyl-pyridin-3-yloxy)-phenylamino]-quinazolin-6-yl}-allyl)-acetamide and isolating the E-N-(3-{4-[4-(6-hydroxymethyl-pyridin-3-yloxy)-phenylamino]-quinazolin-6-yl}-allyl)-2-methoxy-acetamide.

In one preferred embodiment the nutrient medium suitable for Streptomyces rimosus is IOWA medium.

The invention also relates to a press for preparing the compounds of the present invention, comprising the step of preparing the compound in vivo (i.e., the compound is produced in the body).

The invention also relates to a process for preparing the compounds of the present invention, comprising the step of preparing the compound synthetically.

This invention also relates to a method for the treatment of abnormal cell growth in a mammal, including a human, comprising administering to said mammal an amount of a compound of the present invention, as defined above, or a pharmaceutically acceptable salt, solvate, hydrate or prodrug thereof, that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system, (CNS), primary CNS lymphoma, spinal axis tumors, brain stem glioma, pituitary adenoma, or a combination of one or more of the foregoing cancers, In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of the present invention, or a pharmaceutically acceptable salt, solvate or prodrug thereof, that is effective in treating abnormal growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a compound of the present invention, as defined above, or a pharmaceutically acceptable salt, solvate or prodrug thereof, that is effective in treating abnormal cell growth and a pharmaceutically acceptable carrier. In one embodiment of said composition, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one of more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of a compound of the present invention, as defined above or a pharmaceutically acceptable salt, solvate or prodrug thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

This invention also relates to a method for the treatment of a disorder associated with angiogenesis in a mammal, including a human, comprising administering to said mammal an amount of a compound of the present invention, as defined above, or a pharmaceutically acceptable salt, solvate or prodrug thereof that is effective in treating said disorder. Such disorders include cancerous tumors such as melanoma: ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of a compound of of the present invention, or a pharmaceutically acceptable salt, solvate or prodrug thereof, and an amount of one or more substances selected from anti-angiogenesis agents, signal transduction inhibitors, and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of the present invention in the methods and pharmaceutical compositions described herein. Examples of useful COX-II inhibitors include CELEBREX™ (celecoxib), Bextra (valdecoxib), paracoxib, Vioxx (rotecoxib), and Arcoxia (etoricoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998) WO 98/34915 (publish Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999)i, WO 99/52889 (published Oct. 21, 1999), WO 99/29667 (published Jun. 17, 1999), PCT International Application) No. PCT/IB98/01113 (filed Jul. 21, 1998), European Patent Application No. 99302232.1 (filed Mar. 25, 1999). Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those, that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:
3- [[4-(4-fluoro-phenoxy)-benzenesulfonyl]-(1-hydroxycarbamoyl-cyclopentyl)-amino]-propionic acid;
3exo-3-[4-fluoro-phenoxy)-benzenesulfonylamino]-8oxa-bicyclo[3.2.1]octane-3-carboxylic acid hydroxyamide;
(2R, 3R) 1-[4-2-chloro-4-fluoro-benzyloxy)-benzenesulfonyl]-3-hydroxy-3-methyl-piperidine-2-carboxylic acid hydroxyamide;
4-[4-(4fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R, 3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-(4-fluoro-phenoxy)-benzenesulfonyl]-4-hydroxycarbamoyl-tetrahydro-pyran-4-yl)-amino]-propionic acid; acid hydroxyamide;
3-exo-3-[4-(4-chloro-phenoxy)-benzenesulfonylamino]-8-bicyclo[3.2.1]octane-3-carboxylic acid hydroxyamide;
3-endo-3-[4-(4-fluoro-phenoxy-benzenesulfonylamino]-8-oxa-bicyclo-[3.2.1]octane-3-carboxylic acid hydroxyamide; and
3-[4-(4-fluoro-phenoxy)-benzenesulfonylamino]-tetrahydro-furan-3-carboxylic acid hydroxyamide;

and pharmaceutically acceptable salts, solvates and prod rugs of said compounds.

VEGF inhibitors, for example, SU-11248, SU-5416 and SU-6668 (Sugen Inc. of South San Francisco, Calif., USA), can also be combined with a compound of the present invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,888,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22595 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM1862 (Cytran Inc. of Kirkland, Wash., USA); Avastin, an anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Texas, USA) and 2B-1 (Chiron), may be administered in combination with a compound of the present invention. Such erbB2 inhibitors include Herceptin, 2C4, and pertuzumab. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,537,458 (issued Dec. 24, 199), and U.S. Pat. No. 5,877,305 (issued Mar. 2, 1999), each of which is herein incorporated by reference in its entirety, ErbB2 receptor inhibitors useful in the present invention are also described in U.S. Provisional Application No. 60/117,341, filed Jan. 27, 1999, and in U.S. Provisional Application No. 60/117,346, fled Jan. 27, 1999, both of which are herein incorporated by reference in their entirety. Other erbb2 receptor inhibitors include TAK-165 (Takeda) and GW-572016 (Glaxo-Wellcome).

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent application Ser. Nos. 09/221946 (filed Dec. 28, 1998); 09/45058 (filed Dec. 2, 1999); 09/501163 (filed Feb. 9, 2000); 09/539930 (filed Mar. 31, 2000); 09/202796 (filed May 22, 1997); 09/384339 (filed Aug. 26, 1999); and 09/383755 (filed Aug. 26, 1999); and the compounds disclosed and claimed in the following U.S. provisional patent applications: 60/168207 (filed Nov. 30, 1999); 60/170119 (filed Dec. 10, 1999); 60/177718 (filed Jan. 21, 2000), 60/168217 (filed Nov. 30, 1999), and 60/200834 (filed May 1, 2000). Each of the foregoing patent applications and provisional patent applications is herein incorporated by reference in their entirety.

A compound of the present invention may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Provisional Application 60/113,647 (filed Dec. 23, 1998) which is herein incorporated by reference in its entirety.

A compound of the present invention may be applied as a sole therapy or may involve one or more other anti-tumor substances, for example those selected from, for example, mitotic inhibitors, for example vinblastine: alkylating agents, for example cis-platin, oxaliplatin, carboplatin and cyclophosphamide;, anti-metabolites, for example 5-fluorouracil, capecitabine, cytosine and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon, and anti-hormones for example anti-estrogens such as Nolvadex (tamoxifen) or, for example anti-androgens such as Casodex (4′-cyano-3-(4-fluorophenylsulphonyl-2-hydroxy-2-methyl-3′-(trifluoromethyl)propionanilide).

The compounds of the present invention may be used alone or in combination with one or more of a variety of anti-cancer agents or supportive care agents. For example, the compounds of the present invention may be used with cytotoxic agents, e.g., one or more selected from ,he group consisting of a camptothecin, iriootecan HCl (Camptosar), edotecarin, SU-11248, epirubicin (Elience), docetaxel (Taxotere), paclitaxel, rituximab (Rituxan) bevacizumab (Avastin), inatinib mesylate (Gleevac), Erbitux, gefitinib (Iressa), and combinations thereof. The invention also contemplates the use of the compounds of the present invention together with hormonal therapy, e.g., exemestane (Aromasin), Lupron, anastrozole (Arimidex), tamoxifen citrate (Nolvadex), Trelstar, and combinations thereof. Further, the invention provides a compound of the present invention alone or in combination with one or more supportive care products, e.g., a product selected from the group consisting of Filgrastim (Neupogen), ondansetron (Zotran), Fragmin, Procil, Aloxi, Emend, or combinations thereof. Such conjoint treatment may be achieved by way of the simultaneous, sequential or separate dosing of the individual components of the treatment.

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the compounds of the invention.
Alkylating agents include, but are not limited to, nitrogen mustard N-oxide, cyclophosphamide, ifosfamide, melphalan, busulfan, mitobronitol, carboquone, thiotepa, ranimustine, nimustine, or temozolomide;
Antimetabolites include but are not limited to, methotrexate, 6-mercaptopurine riboside, mercaptopurine, 5-fluorouracil (5-FU) alone or in combination with leucovorin, tegafur, UFT, doxifluridine, camofur, cytarabine, cytarabine ocfosfate, enocitabine, S-1, gemcitabine, or fludarabine;
Antibiotics include but are not limited to, actinomycin D, doxorubicin, daunorubicin, neocarzinostatin, bleomycin, peplomycin, mitomycin C, aclarubicin, pirarubicin, epirubicin, zinostatin, stimalamer, or idarubicin;
Plant-derived antitumor agents include but are not limited to, vincristine, vinblastine, vindeshine, etoposide, sobuzoxane, docetaxel, paclitaxel, or vinorelbine;
Platinum-coordinated compounds include but are not limited to, cisplatin, carboplatin, nedaplatin, or oxaliplatin;
Camptothecin derivatives include but are not limited to camptothecin, 10-hydroxycamptothecin, 9-aminocamptothecin, irinotecan, SN-38, edontcarin, and topotecan;
Tyrosine kinase inhibitors are Iressa or SU15416;
Antibodies include Iressa, Erbitux, Avastin, or Rituximab;
Interferons include interferon alpha, interferon alpha-2a, interferon, alpha-2b, interferon beta, interferon gamma-1a or interferon gamma-n1;
Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, or ubenimex; and
Other antitumor agents include mitoxantrone, I-asparaginase, procarbazine, dacarbazine, hydroxycarbamide, pentostatin, or treotinoin.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinase activation; (5) any tumors that proliferate by aberrant serine/threonine kinase activation, and (6) benign and malignant cells of other proliferative diseases in which aberrant serine./threonine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “halo” as used herein, unless otherwise indicated, includes fluoro, chloro, bromo or iodo. Preferred halo groups are fluoro and chloro.

The term “alkyl”, as used herein, unless otherwise indicated, includes saturated monovalent hydrocarbon radicals having straight, cyclic (including mono or multi-cyclic moieties) or branched moieties. It is understood that for said alkyl group to include cyclic moieties it must contain at least three carbon atoms.

The term “cycloalkyl”, as used herein, unless otherwise indicated, includes saturated monovalent hydrocarbon radicals having cyclic (including mono- or multi-cyclic) moieties.

The term “alkenyl”, as used herein, unless otherwise indicated, includes alkyl groups, as defined above, having at least one carbon-carbon double bond.

The term “alkynyl”, as used herein, unless otherwise indicated, includes alkyl groups, as defined above, having at least one carbon-carbon triple bond.

The term “aryl”, as used herein, unless otherwise indicated, includes an organic radical derived from an aromatic hydrocarbon by removal of one hydrogen, such as phenyl or naphthyl.

The term “alkoxy”, as used herein, unless otherwise indicated, includes —O-alkyl groups wherein alkyl is as defined above.

The term “Me” means methyl, “Et” means ethyl, and “Ac” means acetyl.

The term “glucuronic acid” is a substituent that is transferred to a metabolite or transferred to a parent compound to form a metabolite from the phase II conjugation reaction of glucuronidation. Glucuronic acid reacts with an acid or alcohol or phenol moiety on the metabolite or parent compound to form the “glucuronide.”

The term “sulfuric acid” is the substituent that is transferred to a metabolite or transferred to a parent compound to form a metabolite from the conjugation reaction of sulfation. Sulfuric acid reacts with an alcohol or phenol moiety on the metabolite or parent compound to form the “sulfate” or the “sulfate conjugate.”

The phrase “pharmaceutically acceptable salt(s)”, as used herein, unless otherwise indicated, includes salts of acidic or basic groups ,which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

The phrase “substantially pure”, as used herein, unless otherwise indicated, refers to purity of chemical compounds wherein the said compounds are at least 90%, and in one embodiment at least 95%, and in one embodiment at least 99% pure.

Those compounds of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include but are not limited to moieties described in Patini et al., Chem. Rev, 1996, 96, 3147-3176 and references cited therein.

The compounds of the present invention may have asymmetric centers and therefore may exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of the present invention may also exist as tautomers. This invention relates to the use of all such tautomers and mixtures thereof.

The subject invention also includes isotopically-labeled compounds, and the pharmaceutically acceptable salts, solvates and prodrugs thereof, which are identical to those recited in formula 1 or formula 5, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C,¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating bacterial infections through administering prodrugs of compounds of the present invention. Compounds of the present invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a fee amino, hydroxy or carboxylic acid group of compounds of the present invention. The amino acid residues include but are not limited to the 20 naturally occurring amino acids commonly designated by three letter symbols and also includes 4-hydroxyproline, hydroxylsine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drag Delivery Reviews, 1996, 19 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10 Free amines can also be derivatized as amides, sulfonamides or phosphonamides. All of these prodrug moieties may incorporate groups including but not limited to ether, amine and carboxylic acid functionalities.

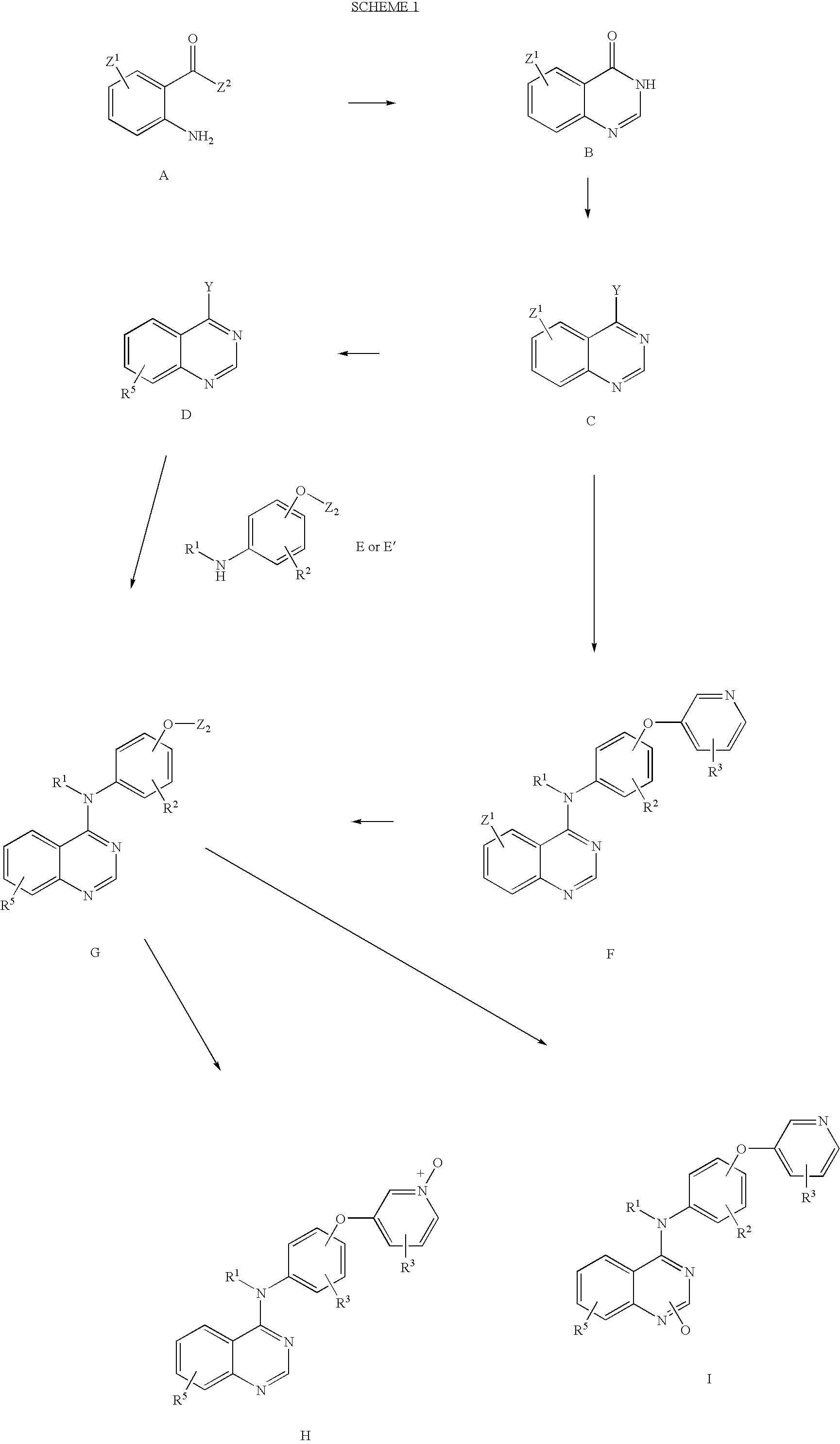

General synthetic methods which may be referred to for preparing the compounds of the present invention are provided in U.S. Pat. No. 5,747,498 (issued May 5, 1998), U.S. patent application Ser. No. 08/953078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30 1979). WO 95/19774 (published Jul. 27, 1995) and WO 97/13771 (published Apr. 17, 1997). Additional procedures are referred to in WO00/44728 (published Aug. 3, 2000), EP 1029853A1 (published Aug. 23, 2000), and WO01/98277 (published Dec. 12, 2001). The foregoing patents and patent applications are incorporated herein by reference in their entirety, Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M. Kazmierczak, F., Leonard, N. J., J. Org. Chem. 1986, 51, 5, p. 616. Palladium catalyzed Heck couplings are described in Heck et. al. Organic Reactions, 1982, 27, 345 or Cabri et, al. in Acc. Chem. Res. 1995, 28, 2.

Starting materials, the synthesis of which is not specifically described above, are either commercially available or can be prepared using methods well known to those of skill in the art.

In each of the reactions discussed or illustrated in the Schemes above, pressure is not critical unless otherwise indicated. Pressures from about 0.5 atmospheres to about 5 atmospheres are generally acceptable, and ambient pressure, i.e., about 1 atmosphere, is preferred as a matter of convenience.

With reference to Scheme 1 above, the compounds of the present invention may be prepared by coupling the compound of formula D wherein R⁵ is defined above, with an amine of formula E or E′ wherein R¹, R², and R³ are as defined above, in an anhydrous solvent, in particular a solvent selected from DMF (N,N-dimethylformamide), DME (ethylene glycol dimethyl ether), DCE (dichloroethane) and t-butanol, and phenol, or a mixture of the foregoing solvents, a temperature within the range of about 50-150° C. for a period ranging from 1 hour to 48 hours. The heteroaryloxyanilines of formula E may be prepared by methods known to those skilled in the art, such as, reduction of the corresponding nitro intermediates. Reduction of aromatic nitro groups may be performed by methods outlined in Brown, R. K., Nelson, N. A. J. Org. Chem. 1954, p. 5149; Yuste, R., Saldana, M, Walls, F., Tet. Lett. 1982, 23, 2, p. 147; or in WO 96/09294, referred to above. Appropriate heteroaryloxy nitrobenzene derivatives may be prepared from halo nitrobenzene precursors by nucleophilic displacement of the halide with an appropriate alcohol as described in Dinsmore, C. J. et. al., Bioorg. Med. Chem. Lett., 7, 10, 1997, 1345; Loupy, A. et. al., Synth. Commun., 20, 18, 1990, 2585; or Brunelle, D. J., Tet. Lett., 25, 32, 1984, 3383. Compounds of formula E in which R¹ is a C₁-C₆ alkyl group may be prepared by reductive amination of the parent aniline with R¹CH(O). The compound of formula D or G may be prepared by treating a compound of formula C or F, wherein Z¹ is an activating group, such as bromo, iodo, —N₂, or —OTf (which is OSO₂CF₃), or the precursor of an activating group such as NO₂, NH, or OH, with a coupling partner, such as a terminal alkyne, terminal alkene, vinyl halide, vinyl stannane, vinylborane, alkyl borane, or an alkyl or alkenyl zinc reagent. Z₂ may be a hydrogen or a protecting group of the phenolic moiety of the aniline or a heteroaromatic piece. The compound of formula C can be prepared by treating a compound of formula B with a chlorinating reagent such as POCl₃, SOCl₂ or ClC(O)C(O)Cl/DMF in a halogenated solvent at a temperature ranging from about 60° C. to 150° C. for a period ranging from about 2 to 24 hours which in turn car be treated with sodium aryloxide in solvent such as aromatic phenols at a temperature ranging from 25 C. to 90 C. In formula C and D, Y is —Cl or —OAr, where Ar is an aryl group, such as phenyl. The heteroaryloxyanilines of formula E′ and quinazoline compounds H (or I) may be prepared by methods similar to above and known to those skilled in the art, with the inclusion of a step of oxidation of the pyridine compounds to the corresponding N-oxide intermediates.

Compounds of the present invention may be converted into another compound of the present invention by standard manipulations to the R⁶ group. These methods are known to those skilled in the art and include a) removal of a protecting group by methods outlined in T. W. Greene and P. G. M. Wuts, “Protective Groups in Organic Synthesis”, Second Edition, John Wiley and Sons, Now York, 1991; and b) displacement of a leaving group (halide, mesylate, tosylate, etc) with a primary or secondary amine, thiol or alcohol to form a secondary or tertiary amine, thioether or ether, respectively.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product, Since a single compound of the present invention may include more than one acidic or basic moieties, the compounds of the present invention may include mono, di or tri-salts in a single compound.

The compounds of the present invention are potent inhibitors of the erbB family of oncogenic and protooncogenic protein tyrosine kinases, in particular erbB2, and thus are all adapted to therapeutic use as antiproliferative agents (e.g., anticancer) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hypetplasia of the prostate (e.g., BPH). It is, in addition, expected that a compound of the present invention may possess activity against a range of leukemias and lymphoid malignancies.

The compounds of the present invention may also be useful in the treatment of additional disorders in which aberrant expression ligand/receptor interactions or activation or signalling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signalling of the erbB tyrosine kinases are involved. In addition, the compounds of the present invention may have therapeutic utility in inflammatory angiogenic and immunologic disorders involving both identified and as yet unidentified tyrosine kinases that are inhibited by the compounds of the present invention.

The compounds of the present invention may also be useful as biomarkers for the metabolism of E-2-Methoxy-N-(3-{4-[3-methyl-4-(6-methyl-pyridin-3-yloxy)-phenylamino]-quinazolin-6-yl}-allyl)-acetamide and may further be used to determine its rate of absorption and metabolic breakdown in mammals, such as humans.

The in vitro activity of the compounds of the present invention may be determined by the following procedure.

The c-erbB2 kinase assay is similar to that described previously in Schrang et. al. Anal. Biochem. 211, 1993, p 233-239. Nunc MaxiSorp 96-well plates are coated by incubation overnight at 37° C. with 100 mL per well of 0.25 mg/mL Poly (Glu, Tyr) 4:1 (PGT) (Sigma Chemical Co., St. Louis, Mo.) in PBS (phosphate buffered saline). Excess POT is removed by aspiration, and the plate is washed three times with wash buffer (0.1% Tween 20 in PBS). The kinase reaction is performed in 50 mL of 50 mM HEPES (pH 7.5) containing 125 mM sodium chloride, 10 mM magnesium chloride, 0.1 mM sodium orthovanadate, 1 mM ATP, 0.48 mg/mL (24 ng/well) c-erbB2 intracellular domain. The intracellular domain or the erbB2 tyrosine kinase (amino acids 674-1255) is expressed as a GST fusion protein in Baculovirus and purified by binding to and elution from glutathione coated beads. The compound in DMSO (dimethylsulfoxide) is added to give a final DMSO concentration of about 25%. Phosphorylation was initiated by addition of ATP (adenosine triphosphate) and proceeded for 6 minutes at room temperature, with constant shaking. The kinase reaction is terminated by aspiration of the reaction mixture and subsequent washing with wash buffer (see above). Phosphorylated PGT is measured by 25 minutes of incubation with 60 mL per well HRP-conjugated PY94 (Oncogene Science Inc. Uniondale, N.Y.) antiphosphotyrosine antibody, diluted to 0.2 mg/mL in blocking buffer (3% BSA and 0.05% Tween 20 in PBS). Antibody is removed by aspiration, and the plate is washed 4 times with wash buffer. The colormetric signal is developed by addition of TMB Microwell Peroxidase Substrate (Kirkegaard and Perry, Gaithersburg, Md.), 50 mL per well, and stopped by the addition of 0.09 M sulfuric acid, 50 mL per well. Phosphotyrosine is estimated by measurement of absorbance at 450 nm. The signal for controls is typically 0.6-1.2 absorbance units, with essentially no background in wells without the POT substrate and is proportional to the time of Incubation for 10 minutes. Inhibitors were identified by reduction of signal relative to wells without inhibitor and IC₅₀ values corresponding to the concentration of compound required for 50% inhibition are determined. The compounds of the present invention exemplified herein may have IC₅₀ values of <10 μM against erB2 kinase as described in the assay above.

The activity of the compounds of the present invention, in vivo, can be determined by the amount of inhibition of tumor growth by a test compound relative to a control. The tumor growth inhibitory effects of various compounds are measured according to the method of as Corbett T. H. et al., “Tumor induction Relationships in Development of Transplantable Cancers of the Colon in Mice for Chemotherapy Assays, with a Note on Carcinogen Structure”, Cancer Res., 35, 2434-2439 (1975) and Corbett T. H., et al., “A Mouse Colon-tumor Model for Experimental Therapy”, Cancer Chemother. Rep. (Part 2)”, 5, 169-186 (1975), with slight modifications. Tumors are induced in the left frank by subcutaneous (sc) injection of 1-5 million log phase cultured tumor cells (murine FRE-ErbB2 cells or human SK-OV3 ovarian carcinoma cells) suspended in 0.1 ml RPMI 1640 medium. After sufficient time has elapsed for the tumors to become palpable (100-150 mm3 in size/5-6 mm in diameter) the test animals (athymic female mice) are treated with test compound (formulated at a concentration of 10 to 15 mg/ml in 5 Gelucire) by the intraperitoneal (ip) or oral (po) route of administration once or twice daily for 7 to 10 consecutive days. In order to determine an anti-tumor effect, the tumor is measured in millimeters with a Vernier caliper across two diameters to and the tumor size (mm3) is calculated using the formula: Tumor size (mm3)=(length×[width]2)/2, according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). Results are expressed as percent inhibition, according to the formula. Inhibition (%)×(TuW_{control}−TuWₜₑₛₜ)/TuW_{control}×100%. The flank site of tumor implantation provides reproducible dose/response effects for a variety of chemotherapeutic agents, and the method of measurement (tumor diameter) is a reliable method for assessing tumor growth rates.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

Advantageously, the present invention also provides kits for use by a consumer for treating disease. The kits comprise a) a pharmaceutical composition comprising a compound of the present invention and a pharmaceutically acceptable carrier, vehicle or diluent; and b) instructions describing a method of using the pharmaceutical composition for treating the specific disease.

A “kit” as used in the instant application includes a container for containing the separate unit dosage forms such as a divided bottle or a divided Foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is seated against the plastic foil at the face of the fail which is opposite from the direction in which the recesses were formed. As a result, the tablets or capsules are individually sealed or collectively sealed, as desired, in the, recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ,” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds the patient when the next dose is to be taken.

In still another embodiment of the kits, the pharmaceutical composition may also comprise an additional compound that can be used in combination with a compound of the present invention, or the kit may comprise two pharmaceutical compositions: one containing a compound of the present invention and another containing an additional compound that can be used in combination with a compound of the present invention.

The active compound may be applied as a sole therapy or may involve one or more other anti-tumor substances, for example those selected from, for example, mitotic inhibitors, for example vinblastine; alkylating agents, for example cisplatin, carboplatin and cyclophosphamide; anti-metabolites, for example 5fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential, or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Administration of a combination of a compound of the present invention and an additional compound or additional compounds means that these compounds can be administered together as a composition or as part of the same unitary dosage form or in separate dosage forms, administered at the same time, or at different times.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coring matters or dyes and, if desired, emulsifying agents or suspending agents, together with diluents such as water, ethanol, propylene glycol, glycerin or combinations thereof.

Methods of preparing various pharmaceutical compositions with a specific amount of active compound are known, or will be apparent, to those skilled in this art. For examples, see Remington's Pharmaceutical Science, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, it was performed using a Waters Alliance HPLC system (2690+996 photodiode array). Preparative HPLC was performed using a Waters 717 autosampler, 996 PDA, 600 controller. Other details regarding the chromatographic procedures are provided within the examples below.

The compounds of the present invention may be synthetically prepared according to the scheme 1 shown above or alternatively they may be prepared using biotransformation techniques well known to those of ordinary skill in the art and as described below.

### EXAMPLES

### Example 1

General Method for Biotransformation

A biotransformation can be achieved by those skilled in the art by contacting the substance to be transformed, and other necessary reactants with a variety of living microorganisms or the enzymes derived from them under conditions suitable for a chemical interaction to occur. Subsequently, the products of the reaction are separated and those of interest are purified for elucidation of their chemical structure and physical and biological properties. The enzymes can be present as: purified reagents, in crude extracts or lysates, in intact cells, in solution, in suspension, as covalently attached to a supporting surface, or imbedded in a permeable matrix (e.g., agarose or alginate beads). The substrate and other necessary reactants (e.g., water, air, cofactors) are supplied as the chemistry dictates.

Generally, the reaction is carried out in the presence of one or more liquid phases, aqueous and/or organic, to promote mass transfer of the reactants and products. The reaction may or may not be conducted aseptically. The conditions for monitoring the progress of the reaction and the isolation of the products of the reaction will vary according to the physical properties of the reaction system and the chemistry of tie reactants and products, and such variations will be appreciated by those of ordinary skill in the art.

The following are two examples of a laboratory-scale method for carrying out aerobic biotransformation that can be exercised by those skilled in the art to produce compounds of interest. Nutrient medium (e.g., IOWA Medium: dextrose, yeast extract, dipotassium hydrogen phosphate, sodium chloride, soybean flour, water; adjusted to neutral pH) is added to one or more culture vessels (e.g., fermentation tubes or flasks) and then steam-sterilized. Each vessel is aseptically inoculated with growth from an agar culture, a suspension of washed cells or spores, or broth from a liquid nutrient medium culture of the biotransforming microorganism. The vessels are mounted on a shaker designed for fermentation and shaken (e.g., rotary operation at 100-300 rpm) at an appropriate temperature (e.g., 20-40° C.) long enough to promote the growth of the microorganisms to a suitable population size (e.g., 1-3 days). The parent compound to be transformed (i.e., substrate) is dissolved in water or a suitable water-miscible solvent (e.g., dimethylsulfoxide, dimethylformamide, ethyl alcohol, methyl alcohol). To each of the biotransformation vessels, the resulting solution is aseptically added to achieve the desired concentration of substrate (e.g., 0.1-02 mg/mL). The dosed vessels are mounted on the shaker and shaken as before, until the substrate has been converted to product(s) by microbial metabolism (e.g.) 1-10 days). The contents of the biotransformation vessel can be mechanically treated (e.g., by filtration or centrifugation) to separate undissolved solids and cells from the aqueous phase or extracted at a pH optimal for extraction of the desired compounds (water-immiscible organic solvents include, but are not limited to, methylene chloride or ethyl acetate). If separated, the solids can be extracted with a suitable water-miscible organic solvent (e.g., methanol). The solvent extract of the solids and the aqueous phase content from the vessels are recovered, combined, and concentrated using suitable methods, e.g., solid phase extraction and drying under reduced pressure. The dried crude is redissolved in a solvent that is compatible with the purification method (e.g., acetonitrile, methanol, water, or HPLC mobile phase). Isolation and purification of the biotransformation product(s) can be achieved by, but not limited to, solid phase extraction (SPE) followed by reversed phase high performance liquid chromatography (HPLC).

The biotransformation product(s) an be monitored during chromatographic, separation for example by UV-absorbance and photodiode array spectral profile. Fractions of the HPLC mobile phase containing the product(s) of interest are retained and the product(s) is/are extracted from the mobile phase using suitable methods, e.g., vacuum drying followed by SPE or water-immiscible organic solvent extraction at a pH optimal for extraction of the desired compounds. The solvent eluate from SPE extraction is recovered, filtered to remove solids, and concentrated under reduced pressure to produce dried purified biotransformation product(s). The chemical structure of the isolated product(s) is determined by mass spectroscopy (MS) and nuclear magnetic resonance (NMR).

### Example 2

### Preparation of E-N-3-{4-[3-Hydroxymethyl-4-(6-methyl-pyridin-3-yloxy)-phenylamino]-quinazolin-6-yl}-allyl)-2-methoxy-acetamide by microbial biotransformation

Fifty milliliters (50 mL) of IOWA Medium (anhydrous dextrose, 20 g; yeast extract, 5 g; dipotassium hydrogen phosphate, 5 g; sodium chloride, 5 g; soybean flour, 5 g; distilled water, 1 L; adjusted to pH 7.2 with 1N hydrochloric acid) were added to each of twenty-nine 250 mL Erlenmeyer flasks with foam plug closures and were steam-sterilized for 20 minutes at 15 psig and 121° C. Three flasks were aseptically inoculated with 0.5 mL of a cryogenically stored (−80° C.) stock of Streptomyces albulus (ATCC 12757) mycelium. The inoculated flasks were mounted vertically on a rotary shaker (2-inch throw) and shaken at 210 rpm and 29° C. for 2 days (inoculum stage). Then 5 mL of the inoculum stage culture was aseptically transferred to each of the remaining 26 flasks (biotransformation stage). The inoculated biotransformation flasks were mounted vertically on a rotary shaker (2-inch throw) and shaken at 210 rpm and 29° C. for 2 days. The methanesulfonate salt of 2-Methoxy-N-(3-{-[3-methyl-4- (6-methyl-pyridin-3-yloxy)-phenylamino]-quinazolin-6-yl}-allyl)-acetamide (i.e. substrate) was dissolved in dimethylsulfoxide (10 mg/mL). To each of the twenty-six biotransformation flasks, 0.5 mL of the resulting solution was aseptically added to give an initial substrate concentration of 0.1 mg/mL (130 mg total in 26 flasks). The dosed flasks were remounted vertically on the rotary shaker and shaken at 210 rpm and 29° C. for an additional 3 days. At the end of the 3-day biotransformation period, contents of the biotransformation flasks were combined. The pH of the whole broth was adjusted to 8.5 with 1N sodium hydroxide. The resulting broth was extracted twice with an equal volume of ethyl acetate. The organic phase was concentrated using a rotary evaporator (40° C. water bath) then taken to dryness under reduced pressure (Savant Speedvac, low heat, full vacuum setting). Dimethylsulfoxide (0.4 mL) was added to this residue which was then loaded onto a preconditioned Waters C18 SepPak (5 g) for solid phase extraction (the cartridge was preconditioned as per manufacturer's directions). After loading, the column was washed with 24 mL distilled water followed by 24 mL of 25% methanol in water, then 24 mL of 50% methanol in water to remove unwanted material. The compound of interest was eluted with 24 mL 75% methanol in water. The 75% methanol in water fractions were dried under reduced pressure (Savant Speedvac, low temperature setting, full vacuum setting) overnight, Methanol was added to each of the tubes, Combining the residues (approximately 0.6 mL) for reversed phase high performance liquid chromatography (HPLC Method 1) to isolate the compound of interest.

HPLC Method 1

| | |
|---|---|
| Column: | Waters SymmetryPrep C18 5μ 19 × 300 mm. |
| Mobile | linear gradient from 0-20 min. |
| phase: | 90:10 to 50:50 over 20 minutes; step to 10:90 at 20.5 min; |
| | hold at 10:90 for 7 minutes |
| | (aqueous buffer [5 mM ammonium acetate, pH 4.5]: |
| | acetonitrile) |
| Flow rate: | 12 mL/min. |
| Monitor: | UV absorbance at 254 nm; photodiode array at 200-400 nm |
| Run Time: | 27 min. |

The title compound had a retention time of approximately 17.2 minutes. HPLC fractions containing the title compound were collected. The pH of the eluate was adjusted to −8.6 with 1N NaOH then extracted twice with an equal volume of dichloromethane. An aliquot of the organic phase was taken to dryness under a stream of nitrogen gas (40° C. water bath) and resuspended in methanol for reversed phase high performance liquid chromatography (HPLC Method 2) for analysis, The compound of interest had a retention time of approximately 14.7 minutes in this analytical assay. The parent compound eluted at approximately 19.3 minutes in the same assay.

HPLC Method 2

| | |
|---|---|
| Column: | Waters Symmetry C18 5μ; 2.1 × 150 mm. |
| Mobile | linear gradient from 0-20 min.; |
| phase: | 90:10 to 50:50 over 20 minutes; step to 10:90 at 20.5 min; |
| | hold at 10:90 for 7 minutes; |
| | (aqueous buffer [5 mM ammonium acetate, pH 4.5]: |
| | acetonitrile) |
| Flow rate: | 0.3 mL/min. |
| Monitor: | UV absorbance at 254 nm; photodiode array at 200-400 nm |
| Run Time: | 30 min. |

The remaining organic phase was concentrated using a rotary evaporator under reduced pressure (40° C. water bath) then taken to dryness under reduced pressure (Savant Speedvac, low temperature setting, full vacuum setting). The compound of interest (15.6 mg) was isolated as a yellow powder.

It had UV-light absorbance maxima (λₘₐₓ) at 242.6 nm, 312.5 nm, and 347 nm. Mass spectrometry; m/z=486.5.

¹H (CD₃OD) δ 8.78 (s, 1H), 8.65 (d, J=1.6 Hz, 1H), 8.45 (d, J=2.8 Hz, 1H), 8.23 (dd, J=8.7, 1.6 Hz, 1H), 8.02 (d, J=2.8 Hz, 1H), 7.98 (dd, J=8,7, 2.8 Hz, 1H), 7.85 (d, J=8.7 Hz, 1H), 7.81 (dd, J=8.7, 2.8 Hz, 1H), 7.78 (d, J=8.7, 1H), 7.21 (d, J=8.7 Hz, 1H) 6.78 (d, J=15.9 Hz, 1H), 6.64 (dt, J=15.9, 5.6 Hz, 1H), 4.74 (s, 2H), 4.14 (dd, J=5.6, 1.2 Hz, 2H), 3.98 (s, 2H), 3.48 (s, 3H), 2.73 (s, 3H). ¹³C (CD₃OD) δ 171.6, 161.2, 160.9, 160.5, 154.8, 151.1, 150.4 150.0, 139.0, 137.9, 134.8, 134.6, 134.3, 132.9, 132.7, 130.8, 128.9, 128.2, 125.9, 125.7, 121.2, 120.0, 119.9, 114.3, 71.7, 58.8, 58.7, 40.6, 18.9.

### Example 3

### Preparation of E-N-(3-{4-[-(6-hydroxymethyl-pyridin-3-yloxy)-3-methyl-phenylamino]-quinazolin-6-yl}-allyl)-2-methoxy-acetamide

The compound of interest E-N-(3-{4-[4-(6-hydroxymethyl-pyridin-3-yloxy)-3-methyl-phenylamino]-quinazolin-6-yl}-allyl-2-methoxy-acetamide was prepared using the procedure described in Example 2 with the following differences as noted below:

The microorganism used was Streptomyces rimosus (ATCC 23955) mycelium (instead of Streptomyces albulus (ATCC 12757) mycelium). The dosed flaskes were shaken for an additional 5 days (instead of 3 days). The title compound had a retention time of approximately 18.5 minutes, using HPLC Method 1 of Example 2. After the HPLC fractions (from HPLC Method 1) were collected, the eluate was then extracted twice with an equal volume of dichloromethane (no pH adjustment of eluate was carried out). The compound of interest had a retention time of approximately 15.3 minutes according to the second high performance liquid chromatography (HPLC Method 2). The parent compound eluted at approximately 19.3 minutes in the same assay. The compound of interest (10.4 mg) was isolated as a yellow powder.

The compound had UV-light absorbance maxima (λₘₐₓ) at 242.6 nm, 312.5 nm, and 347 nm. Mass spectrometry: m/z=486.5.

¹H (CD₃OD) δ 8.53, (s, 1H), 8.40 (d, J=1.2 Hz, 1H), 8.22 (d, J=2.8 Hz, 1H), 8.03 (dd, J=8.7, 2.0 Hz, 1H), 7.72 (d, J=2.8 Hz, 1H), 7.76 (d, J=8.7 Hz, 1H), 7.64 (dd, J=8.7, 2.8 Hz, 1H), 7.54 (d, J=8.7 Hz, 1H), 7.41 (dd, J=8.7, 2.8 Hz, 1H), 7.04 (d, J=8.7 Hz, 1H), 6.76 (d, J=15.9 Hz, 1H), 6.53 (dt, J=15.9, 5.6 Hz, 1H), 4.70 (s, 2H), 4.12 (m, 2H), 3.99 (s, 2H), 3.48 (s, 3H), 2.29 (s, 3H). ¹³C (CD₃OD) δ 171.6, 159.3, 155.1, 154.3, 153.7, 151.2, 147.1, 138.0, 136.7, 135.3, 131.9, 130.7, 130.1, 128.5, 127.0, 126.0, 125.4, 123.1, 122.2, 120.4, 120.3, 115.5, 71.7, 64.2, 58.6, 40.6, 15.4.

### Example 4

### Preparation of E-3-{4-[3-Methyl-4-(6methyl-pyridin-3-yloxy)-phenylamino]-quinazolin-6-yl}-acrylic acid

To a cooled (0° C.) stirred suspension of sodium hydride (NaH) (4.8 g, 60%, 0.12 mol) in anhydrous DMF (60 ml) was added dropwise a solution of phenol (PhOH) (11.3 g, 0.12 mol) in dry DMF (50 mL). After the addition, 4-chloro-6-iodoquinazolinone (29 g, 0.1 mol) was added portionwise. Thereafter the tooling bath was removed and the resulting solution was stirred at room temperature for 1.5 h, quenched with water (300 mL). The product precipitated and was extracted with ethyl acetate (EtOAc) (400 mL). The separated organic layer was washed with aq. sodium hydroxide NaOH, water, brine, dried over N12SO4 and concentrated to give 6-iodo-4-phenoxyquinazoline as off-white solid (32.9 g, 94%). Crystallizaton from EtOAc gave white soft and short needle crystals.

A mixture of 6-iodo-4-phenoxyquinazoline (3.5 g, 10 mmol) as prepared in the preceding paragraph, methyl acrylate (6 g, 70 mmol). Pd(OAc)₂ (140 mg, 0.62 mmol) and Ph₃P (320 mg, 1.22 mmol) in triethylamine (Et₃N)/DMF was purged with N₂ and the pressure reaction vessel was tightly capped. The reaction was then heated on an oil-bath at 110° C. with stirring. Thin layer chromatography indicated that the reaction was complete after 3 hours. The product mixture was then transferred to a round bottomed task and purged with a stream of N₂ to remove the methyl acrylate. The residue was then dissolved in ethyl acetate, washed with water, brine, dried over sodium sulfate and concentrated to give crude E-3-(4-Phenoxy-quinazolin-6-yl)-acrylic acid methyl ester as yellow solid which was recrystallized from ethyl acetate to yield 2.3 g (70%) of pale yellow crystalline solid in two crops.

A mixture of the product (E-3-(4-Phenoxy-quinazolin-6-yl)-acrylic acid methyl ester) from the preceding paragraph (307 mg, 1 mmol) and the desired aniline (215 mg, 1 mmol) was taken up in phenol (2 g) and the resultant mixture heated at 100° C. on an oil-bath and a clear solution was obtained. After heating 20 h, the brown solution was distilled under reduced pressure to remove phenol. The residue was partitioned between dilute NaOH and methylene chloride. The separated organic layer was washed with brine, dried over sodium sulfate and concentrated to give the crude product, which was purified by chromatography to give pure E-3-{4-[3-Methyl-4-(6-methyl-pyridin-yloxy)-phenylamino]-quinazolin]-yl}-acrylic acid methyl ester (480 mg).

A mixture of the methyl ester (450 mg, 1 mmol) and LiOH.H₂O (0.63 g, 15 mmol) in methanol/water (10/1 ml) was refluxed for 3 h. After cooling the reaction was neutralized with acetic acid (0.9 g, 15 mmol) in 2 mL of H₂O to pH 6.0. A dear solution was obtained initially and the acid product precipitated later as a yellow solid, which was collected by vacuum filtration and dried to give the final product E-3-{4-[3-Methyl-4-(6-methyl-pyridin-3-yloxy)-phenylamino]-quinazolin-6-yl}-acrylic acid as a yellow solid (280 mg, 68%).

¹H (CD₃OD): δ 2.24 (s, 3H), 2.48 (s, 3H), 6.70 (d, J=16 Hz, 1H), 6.98 (d, 1H), 7.28 (m, 2H), 7.6 (m, 1H), 7.69 (m, 1H), 7.76 (m, 1H), 7.78 (d, J=16 Hz, 1H, 8.1 (m, 2H), 8.5 (s, 1H), 8.6 (d, 1H). m/z (ES+) (M+1) 413.4. HPLC Rt=4.831 mins.

### Example 5

### Identification of cysteine conjugates of E-N-(3-{4-[4-(6-hydroxymethyl-pyridin-3-yloxy)-3-methyl-phenylamino]-quinazolin-6-y}-allyl)-2-methoxy-acetamide

A group of two male and two female bile-duct cannulated Sprague-Dawley rats (211-252 g) were housed individually in stainless steel metabolic cages and were administered a single 50 mg/kg oral dose of [¹⁴C] E-N-(3-{4-[4-(6-hydroxymethyl-pyridin-3-yloxy)-3-methyl-phenylamino]-quinazolin-6-yl}-allyl)-2-methoxy-acetamide (“the parent compound”) (223 μCi/kg) in 0.5% aqueous methylcellulose. Urine and bile were collected at 0-8, 8-24 and 24-48 hours post dose. The biological samples were stored at −20° C. until analysis.

Urine samples (−10 ml) were flash frozen in 20 ml flasks and lyophilized overnight. Sample residues were reconstituted in 0.4 ml of water and 0.2-0.5 ml of MeOH. These solutions were vortexed for about 1 min, transferred to 1.5 ml eppendorf tubes and then centrifuged at 14,000 rpm for about 2 min. Aliquots (100 μl) of the supernatants were injected onto the HPLC column without further purification. Bile samples were thawed at room temperature and pooled from 0-48 hours. The pooled samples (5 mL) were diluted with 15 ml acetonitrile. The mixtures were sonicated and vortexed for 10 min then centrifuged at 3000 rpm for 15 min. The supernatants were separated and were evaporated to dryness under N2 in a TurboVap at 40 C. Samples were reconstituted in 0.4 mL of acetonitrile/water (1:1). Aliquots 100 μL were injected onto the HPLC column for LC/MS analysis.

Metabolite quantification was performed by measuring the areas of individually separated HPLC peaks detected using a radioactive monitor (β-RAM, IN/US, Tampa, Fla.) (“β-RAM”). The β-RAM provided integrated peak representation printouts in CPM as well as the percentage of the radiolabelled material. The β-RAM was operated in the homogeneous liquid scintillation counting mode with the addition of 3 ml/min of True-Count (IN/US) scintillation cocktail to the effluent post-MS detection.

High Performance Liquid Chromatography

The HPLC system consisted of a HP-1050 solvent delivery system, a HP-1050 membrane degasser an HP-1050 autoinjector (Hewlett Packard, Palo Alto, Calif.), a Thermo Separations spectromonitor 3200 UV and a radioactive monitor (β-RAM; IN/US, Tampa. Fla.). Chromatography was performed on a Kromasil C-18 column (4.6 mm×150 mm, 5 μm) with a mobile phase containing a mixture of 10 mM ammonium formate (pH=3.0; solvent A) and acetonitrile (solvent B). The flow rate was 1.0 mL/min and the separation was achieved at ambient temperature using the following gradient program:

| Time (min) | Solvent A | Solvent B |
|---|---|---|
| 0 | 95 | 5 |
| 20 | 80 | 20 |
| 37 | 75 | 25 |
| 40 | 65 | 35 |
| 42 | 20 | 80 |
| 45 | 95 | 5 |
| 50 | 95 | 5 |

The system was allowed to equilibrate for 5 min prior to the next injection.

Identification of the metabolites was performed on a Finnigan LCQ Deca LC/MS/MS equipped with an electrospray ion source. The effluent from the HPLC column was split, and about 50 μl/min was introduced into the API source. The remaining effluent was directed into the flow cell of the β-RAM. The β-RAM response was recorded in real time by the mass spectrometer data system, which provided simultaneous detection of radioactivity and mass spectrometry data. The electrospray interface was operated at 4500V and the mass spectrometer was operated either in the positive ion or negative ion mode.

The metabolites had a retention time of −27.5 min on HPLC. A full scan MS revealed a protonated molecular ion at m/z 589, 119 amu higher than the parent compound, suggesting that it was a conjugate. Its MS² product ion spectrum showed fragments at m/z 528, 502, 500, 468, 456, 413 and 379. The ion at m/z 500 resulted from loss of the methoxyacetamide moiety from the protonated molecular ion. The ions at m/z 502 and 468 likely resulted from elimination of the amino acrylic acid and cysteine moiety, respectively from the parent compound. Based on these data, 3-((-3-(2-methoxyacetamido)-1-(4-(4-(6-methylpyridin-3-yloxy)-3-methylphenylamino)quinazolin-6-yl)prop-1-enyl)sulfanyl)-2-aminopropanoic acid and 3-((-3-(2-methoxyacetamido)-1-(4-(4-(6-methylpyridin-3-yloxy)-3-methylphenylamino)quinazolin-6-yl)prop-1-en-2-yl)sulfanyl)-2-aminopropanoic acid and mixtures thereof were tentatively identified as a cysteine conjugates of E-N-(3-{4-[4-(6-hydroxymethyl-pyridin-3-yloxy)-3-methyl-phenylamino]-quinazolin-6-yl}-allyl)-2-method-acetamide.

### Example 6

### Identification of N-acetyl-cysteine conjugate of E-N-(3-{4-[4-(6-hydroxymethyl-pyridin-3-yloxy)-3-methyl-phenylamino]-quinazolin-6-yl}-allyl)-2-methoxy-acetamide

The compounds of interest, 3-((-3-(2-methoxyacetamido)-1-(4-(4-(6-methylpyridin-3-yloxy)-3-methylphenylamino)quinazolin-6-yl)prop-1-enyl)sulfanyl)-2-acetylpropanoic acid and 3-((-3-(2-methoxyacetamido)-1-(4-(4-(6-methylpyridin-3-yloxy)-3-methylphenylamino)quinazolin6-yl)prop-1-en-2yl)sulfanyl)-2-acetylpropanoic acid and mixtures thereof, were identified using the procedure described in Example 5. The compounds of interest had a retention time of −30.5 min on HPLC. A full scan MS revealed a protonated molecular ion at m/z 631, 161 amu higher than the parent compound, suggesting that it was a conjugate. Its MS² product ion spectrum showed fragments at m/z 589, 542, 502, 500, 468, 454, 413 and 379. The ion at m/z 542 resulted from loss of the methoxyacetamide moiety from the protonated molecular ion. The ion at m/z 468 likely resulted from elimination of the N-acetyl-cysteine moiety, respectively. Based on these data, 3-((-3-(2-methoxyacetamido)-1-(4-(4-(6-methylpyridin-3-yloxy)-3-methylphenylamino)quinazolin-6-yl)prop-1-enyl)sulfanyl)-2-acetylpropanoic acid and 3((-3-(2-methoxyacetamido)-1-(4-(4-(6-methylpyridin-3-yloxy)-3-methylphenylamino)quinazolin-6-yl)prop-1-en-2-yl)sulfanyl)-2-acetylpropanoic acid and mixtures thereof were tentatively identified as N-acetyl-cysteine conjugate of E-N-(3-(4-[4-(6-hydroxymethyl-pyridin-3-yloxy)-3-methyl-phenylamino]-quinazolin-6-yl)-allyl)-2-methoxy-acetamide.

### Example 7

### Identification of 3-((3-(2-methoxyacetamido)-1-(4-(4-(6-hydroxymethylpyridin-3-yloxy)-3-methylphenylamino)quinazolin-6-yl)prop-1-enyl)sulfanyl)-2-acetylpropanoic acid and 3-((-3-(2-methoxyacetamido)-1-(4-(4-(6-hydroxymethylpyridin-3-yloxy)-3-methylphenylamino)quinazolin-6-yl)prop-1-en-2-yl)sulfanyl)-2-acetylpropanoic acid

The compounds of interest, 3-((-3-(2-methoxyacetamido)-1-(4-(4-(6-hydroxymethylpyridin-3-yloxy)-3-methylphenylamino)quinazolin-6-yl)prop-1-enyl)sulfanyl)-2-acetylpropanoic acid and 3-((-3-(2-methoxyacetamido)-1-(4-(4-(6-hydroxymethylpyridin-3-yloxy)-3-methylphenylamino)quinazolin-6-yl)prop-1-en-2yl)sulfanyl)-2-acetylpropanoic acid and mixtures thereof were identified using the procedure described in Example 5. The compounds of interest had a retention time of −28.4 min on HPLC. A full scan MS revealed a protonated molecular ion at m/z 647, 16 amu higher than the compounds of Example 6, suggesting the addition of oxygen atom to the identified compounds of Example 6. Its MS² product ion spectrum showed fragments at m/z 629, 585, 518, 500, 486, 469, 439 and 411. The ions at m/z 629 and 585 resulted from loss of the water and CO₂ respectively, from the protonated molecular ion at m/z 647. The ion at m/z 518 is likely resulted from elimination of acetyl amino-acrylic acid moiety from the protonated molecular ion. Based on these data, 3-((-3-(2-methoxyacetamido)-1-(4-(4-(6-hydroxymethylpyridin-3-yloxy)-3-methylphenylamino)quinazolin-6-y)prop-1-enyl)sulfanyl)-2-acetylpropanoic acid and 3-((-3-(2-methoxyacetamido)-1-(4-(4-(4-(6-hydroxymethylpyridin-3-yloxy)-3-methylphenylamino)quinazolin-6-yl)prop-1-en-2-yl)sulfanyl)-2-acetylpropanoic acid and mixtures thereof were identified.

### Example 8

### Preparation of 2-Methoxy-N-(3-(4-[3-methyl-4-(6-methyl-1-oxy-pyridin-3-yloxy)-phenylamino]-quinazolin-6-yl]-allyl)-acetamide

To 2-Methoxy-N-(3-[4-[3-methyl-4-(6-methyl-pyridin-3-yloxy)-phenylamino]-quinazolin-6-yl)-allyl)-acetamide (385 mg, 0.82 mmol) in 10 ml CHCl₃ was added mCPBA (77%, 322 mg, 1.4 mmol), stirred at room temperature for 2-3 hours or until HPLC showed the major product is the desired one. The mixture was directly loaded to ISCO™ column and chromatographed by 2% to 10% MeOH in CH₂Cl₂ to yield the desired product (99 mg, 24%). m/z (ES+) (M+1): 502.4, HPLC:Rt=4.320 mins. HNMR: ¹H (CD₃OD): δ 2.23 (s, 3H), 2.45 (s, 3H), 3.44(s, 3H), 3.04(s, 2H), 4.08(d, J=6 Hz, 2H), 6.50(m, 1H), 6.70 (d, J=16 Hz, 1H), 7.10(d, 1H), 7.12(d, 1H), 7.45(d, J=9 Hz, 1H), 7.65 (d, J=9 Hz, 1H) 7.69 (d, J=9 Hz, 1H), 7.72 (s, 1H), 7.95 (m, 1H), 8.05 (s, 1H), 8.32 (s, 1H), 8.46 (s, 1H).

### Example 9

### Preparation of N-(3-[4-{4-(6-Hydroxymethyl-1-oxy-pyridin-3-yloxy)-3-methyl-phenylamino]-quinazolin-6-yl}allyl)-2-methoxy-acetamide

a) Preparation of [5-(2-methyl-4-nitrophenoxy)pyridine-2-yl]methanol

To 2-methyl-5-2-methyl-4-nitrophenoxy)pyridine (5 g, 20.4 mmol) in 100 ml CH₂Cl₂ was added mCPSA (6.88 g, 30.7 mmol). The mixture was stirred at room temperature overnight. Na₂SO₃ (3 g) in 50 mL water was added to the reaction which resulted in an orange mixture. The organic layer was separated and was washed with saturated NaHCO₃ (aq.), brine several times. The mixture was dried over Na₂SO₄, filtered and concentrated. To the residue was added TFAA and 80 ml CH₂Cl₂ and the mixture was stirred at room temperature overnight. The reaction was quenched with MeOH at 0° C. and stirred until bubble ceased. The mixture was concentrated and the residue was diluted with EtOAc. The solution was washed with 10% NaHCO₃ and brine, dried over Na₂SO₄, filtered, and concentrated. The residue was chromatographed by 20-75% EtOAc/Hexane to obtain 3.5 g (66%) desired product. HPLC: Rt=5.171 min.

b) Preparation of [5-(2-methyl-4-nitrophenoxy)-1-oxidopyridin-yl]methanol

To [5-(2-methyl-4-nitrophenoxy)pyridine-2-yl]methanol (1.2 g, 4.6 mmol) in 30 ml CH₂Cl₂ was added mCPBA (77%, 1.03 g, 4.6 mmol). The mixture was stirred at room temperature for 2 hours. White solid precipitation was observed and filtered. The mixture was concentrated and chromatographed by 3-10% MeOH/CH₂Cl₂ to obtain the desired product (1.0 g, 78%), HPLC: Rt=4.269 min.

c) Preparation of [5-(4-amino-2-methylphenoxy)-1-oxidopyridin-2-yl]methanol

A mixture of [5-(2-methyl-4-nitrophenoxy)-1-oxidopyridin-2yl]methanol (1.0 g, 3.6 mmol), PtO2 (40 mg, 0.18 mmol) and 5-10 mL EtOAc was charged 40 PSI H₂ in Parr shaker for 10 mins. The mixture was filtered through celite and concentrated to obtained the desire product (700 mg, 78%). HPLC: Rt=2.812 min.

d) Preparation of (5-{4-[(6-iodoquinazolie-4-yl)amino]-2-methylphenoxy}-1-oxidopyridin-2-yl)methanol

A mixture of 4-chloro-6-iodoquinazoline (374 mg, 1.3 mmol), 5-(4-amino-2methylphenoxy)-1-oxidopyridin-2-yl]methanol (160 mg, 1.3 mmol), 1,2-Dichloroethane (2 mL) and t-BuOH (2 mL) was heated up to 90° C. in a sealed tube for ½ hour. To the mixture was added Et2O and filtered the precipitation) to obtain the desired product (456 mg). m/z (ES+) (M+1) 501.2, HPLC:Rt=5.169 mins.

e) Preparation of N-(3-{4-[4-(6-Hydroxymethyl-1-oxy-pyridin-3-yloxy)-3-methyl-phenylamino]-quinazolin-6-yl}-allyl)-2-methoxy-acetamide

2-methyl-2-butene (0.59 ml, 5.60 mmol, 2.8 equiv.) was added over 1 hour to a cold (0-5° C.) solution of BH₃*THF complex (1.0 M sol, 3.0 ml, 3.0 mmol, 1.5 equiv.) kept under N₂. The reaction mixture was stirred at this temperature for 30 minutes followed by the addition of 2-Methoxy-acetic acid propargylamide (255 mg, 2 mmol, 1.0 equiv). dissolved in dry THF (1 ml) over 15 minutes. The ice-bath was removed and the reaction mixture was warmed to room temperature over 20 minutes. The reaction mixture was then heated at 35° C. for 1 hour. K₂CO₃ (0.55 g, 4 mmol, 2.0 equiv.) dissolved in degassed H₂O (1.2 ml) was added over 30 minutes to the reaction mixture. During the addition of the first half gas evolution was observed which seized during further addition of (5-{4-[(6-iodoquinazolie-4-yl)amino]-2-methylphenoxy}-1-oxidopyridin-2-yl)methanol (3 mmol, 1.5 equiv.) was added in three portions giving a yellow suspension. PPh₃ (21 mg, 0.08 mmol, 4 mol %) and Pd(OAc)₂ (4.5 mg, 0.02 mmol, 1 mol %) were added each in one portion and the reaction mixture was heated to reflux (65-68° C.). After about 30 minutes a yellow solution was obtained and the reaction was monitored by HPLC assay. After 18 hours the reaction mixture was cooled to room temperature followed by the addition of half-saturated NaCl solution (10 ml) and EtOAc (10 ml). The organic phase was separated, washed with H₂O (5 ml) and concentrated at 50° C. and a pressure of less than 200 mbar. Purification by plug filtration, SiO₂, EtOAc/MeOH=9/1. The title compound was obtained as light yellow crystals (0.55 g, 59%). HNMR: ¹H (CD₃OD): δ 2.23 (s, 3H), 3.44(s, 3H), 3.94(s, 2H), 4.08(d, J=5 Hz, 2H), 4.75(s, 2H), 6.50(m, 1H), 6.70 (d, J=16 Hz, 1H), 7.10(d, J=9 Hz, 1H), 7.23(d, J=2 Hz, 1H), 7.61(d, 1H), 7.62(d, 1H), 7.70 (d, 1H), 7.73(s, 1H), 7.95 (d, J=2 Hz, 1H), 8.03 (s, 1H), 8.34 (s, 1H), 8.46 (s, 1H).

The compounds of the present invention can also be produced in mixtures as metabolites of the E-2 -Methoxy-N-(3-{4-[3-methyl-4-(6-methyl-pyridin-3-yloxy)-phenylamino]-quinazolin-6-yl}-allyl)-acetamide whose structure is shown below.

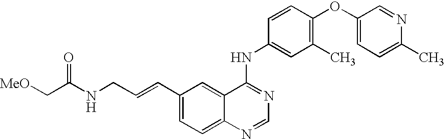

As such, E-2-Methoxy-N-(3-{4-[3-methyl-4-(6-methyl-pyridin-3-yloxy)-phenylamino]-quinazolin-6-yl}-allyl)acetamide can be incubated with mouse, rat, monkey, dog, and human hepatic tissue preparations (slices, homogentates, hepatocytes, microsomes) or with recombinant enzymes (e.g., human CYP containing insect cell microsomes. Bile, urine, and plasma samples can be collected, and further worked up to obtain samples of mixtures of metabolites. These samples can then be subjected to separation by HPLC, and analyzed by standard instrumentation techniques such as mass spectrometry, NMR and UV.

The present invention is not to be limited in scope by the specific embodiments described herein. Indeed, various modifications of tie invention in addition to those described herein will become apparent to those skilled in the art from the foregoing description. Such modifications are intended to fall within the scope of the appended claims.

All patents, applications, publications test methods, literature and other materials cited herein are hereby incorporated by reference in their entireties.

1. A substantially pure compound of the formula 1 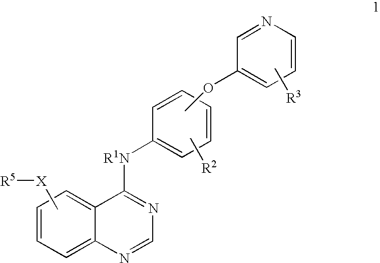
or a pharmaceutically acceptable salt, solvate or prodrug thereof, wherein:
X is selected from the group consisting of a bond, —(CH═CH)— and —(CH₂—CH₂)— wherein any carbon atom in X may be optionally substituted with R⁹;
R¹ is selected from the group consisting of hydrogen and —(C₁-C₅) alkyl;
R² is selected from the group consisting of hydrogen, hydroxyl, —(C₁-C₁₀) alkyl, —(C₁-C₆) alkoxy, and —(C₁-C₆) hydroxyalkyl group;
R³ is selected from the group consisting of hydrogen, —(C₁-C₆) alkyl, —(C₁-C₆) hydroxyalkyl, —CH₂OSO₂OH and —C(O)OR⁴, wherein R⁴ is selected from the group consisting of hydrogen and —(C₁-C₈) alkyl;
R⁵ is selected from the group consisting of —C(O)OH, —C(O)H—(CR⁶R⁷)ₘ—NR¹R⁶, —(CR⁶R⁷)ₘ—OH, —(CR⁶R⁷)ₘ—NR¹C(O)—CO₂H, —(CR⁶R⁷)ₘ—NR¹—C(O)—(CR⁶R⁷)ₘ—OH, and —(CR⁶R⁷)ₘ—NR¹—(C(O))ₙ—(CR⁶R⁷)ₘ—O(C₁-C₆) alkyl, wherein m is an integer from 0 to 3 and wherein n is an integer from 1 to 2; each R⁶ and R⁷ is independently selected from the group consisting of H and C₁-C₆ alkyl, and wherein R⁸ is selected from the group consisting of H, C₁-C₆ alkyl, —C(O)—(C₁-C₆ alkyl) and —C(O)—(CR⁸CR⁷)ₘ—O(C₁-C₆ alkyl);
R⁸ is —S—(CR⁶R⁷)_{q}—CR¹(NR¹R⁸ )—(CR⁶R⁷)ᵣ—C(O)OH, wherein q is an integer from 0 to 3 and wherein r is an integer from 0 to 2;
wherein the compound of formula 1 is further optionally substituted by a hydroxy or an O-glucuronic acid substituent;
and wherein any nitrogen atom in the compound of formula 1 is optionally bound to an oxygen to form an N-oxide moiety (—N→O).

2. A compound of claim 1 further comprising a moiety of formula 2: 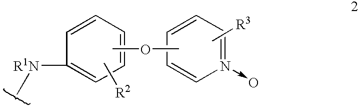
wherein R¹, R³, R⁵, and X are defined above.

3. A compound of claim 1 further comprising a moiety of formula 3: 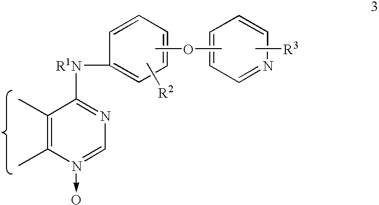
wherein R¹-R³, R⁵ and X are as defined above.

4. A compound of claim 1 further comprising a moiety of formula 4: 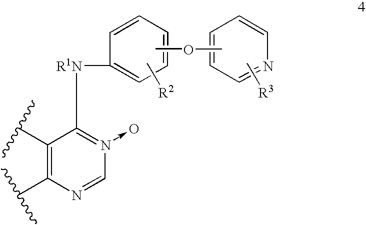
wherein R¹-R³, R⁵ and X are as defined above.

5. A compound of claim 1 selected from the group consisting of:
2-Methoxy-N-(3-{4-[3-methyl-4-(6-methyl-1-oxy-pyridin-3-yloxy)-phenylamino]-quinazolin-6-yl}-allyl)-acetamide;
2-Hydroxy-N-(3-{4-[3-methyl-4-(6-methyl-1-oxy-pyridin-3-yloxy)-phenylamino]-quinazolin-6-yl}-allyl)-acetamide;
3-{4-[3-Methyl-4-(6-methyl-1-oxy-pyridin-3-yloxy)-phenylamino]-quinazolin-6-yl}-prop-2-en-1-ol;
N-(3-{4-[4-(6-Hydroxymethyl-1-oxy-pyridin-3-yloxy)-3-methyl-phenylamino]-quinazolin-6-yl}-allyl)-2-methoxy-acetamide;
and the pharmaceutically acceptable salts, prodrugs, hydrates and solvates of the foregoing compounds.

6. A method for the treatment of abnormal cell growth in a mammal comprising administering to said mammal an amount of a compound of claim 1 that is effective in treating abnormal cell growth.

7. A method according to claim 6, wherein said abnormal cell growth is cancer.

8. A method according to claim 7, wherein said cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, color, cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

9. A method for the treatment of abnormal cell growth in a mammal which comprises administering to said mammal an amount of a compound of claim 1 that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

10. A pharmaceutical composition for the treatment of abnormal cell growth in a mammal comprising an amount of a compound of claim 1 that is effective in treating abnormal cell growth, and a pharmaceutically acceptable carrier.

11. A kit for the treatment of abnormal cell growth comprising a) a pharmaceutical composition comprising a compound of claim 1 and a pharmaceutically acceptable carrier, vehicle or diluent; and b) instructions describing a method of using the pharmaceutical composition for treating the abnormal cell growth.

12. A compound of claim 1 selected from the group consisting of:
3-((-3-(2-methoxyacetamido)-1-(4-(4-(6-methylpyridin-3-yloxy)-3-methylphenylamino)quinazolin-6-yl)prop-1-enyl)sulfanyl)-2-aminopropanoic acid;
3-((-3-(2-methoxyacetamido)-1-(4-(4-(6-methylpyridin-3-yloxy)-3-methylphenylamino)quinazolin-6-yl)prop-1-en-2yl)sulfanyl)-2-aminopropanoic acid;
3-((-3-(2-methoxyacetamido)-1-(4-(4-(6-methylpyridin-3-yloxy)-3-methylphenylamino)quinazolin-6-yl)prop-1-enyl)sulfanyl)-2-acetylpropanoic acid;
3-((-3-(2-methoxyacetamido)-1-(4-(4-(6-methylpyridin-3-yloxy)-3-methylphenylamino)quinazolin-6-yl)prop-1-en-2-yl)sulfanyl)-2-acetylpropanoic acid;
3-((-3-(2-methoxyacetamido)-1-(4-(4-(6-hydroxymethylpyridin-3-yloxy)-3-methylphenylamino)quinazolin-6-yl)prop-1-enyl)sulfanyl)-2-acetylpropanoic acid;
3-((-3-(2-methoxyacetamido)-1-(4-(4-(6-hydroxymethylpyridin-3-yloxy)-3-methylphenylamino)quinazolin-6-yl)prop-1-en-2-yl)sulfanyl)-2-acetylpropanoic acid; and
2-Amino-3-(1-hydroxy-3-{4-[3-methyl-4-(6-methyl-pyridin-3-yloxy)-phenylamino]-quinazolin-6-yl}-propylsulfanyl)-propionic acid
and the pharmaceutically acceptable salts, prodrugs, hydrates and solvates of the foregoing compounds.

13. A substantially pure compound of the formula 5 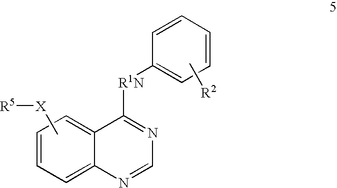
or a pharmaceutically acceptable salt, solvate or prodrug thereof, wherein:
X is selected from the group consisting of —(CH═CH)— and —(CH₂—CH₂)— wherein any carbon atom in X may be optionally substituted with R⁹;
R¹ is selected from the group consisting of hydrogen and —(C₁C₆) alkyl;
R² is selected from the group consisting of hydrogen, hydroxy, —(C₁C₁₀) alkyl, —(C₁-C₆) alkoxy, and —(C₁-C₆) hydroxyalkyl group;
R⁵ is selected from the group consisting of —C(O)OH, —C(O)H —(CR⁶R⁷)ₘ—NR¹R⁸, —(CR⁶R⁷)ₘ—OH, —(CR⁶R⁷)ₘ—NR¹C(O)—CO₂H, —(CR⁶R⁷)ₘ—NR¹—C(O)—(CR⁶R⁷)ₘ—OH, and —(CR⁶R⁷)ₘ—NR¹—(C(O))ₙ—(CR⁶R⁷)ₘ—O(C₁-C₆) alkyl, wherein m is an integer from 0 to 3 and wherein n is an integer from 1-2; each R⁶ and R⁷ is independently selected from the group consisting of H and C₁-C₆ alkyl, and wherein R⁸ is selected from the group consisting of H, C₁-C₆ alkyl, —C(O)C₁-C₆ alkyl) and —C(O)—(CR⁸R⁷)ₘ—(C₁-C₆ alkyl);
wherein the compound of formula 5 is further optionally substituted by a hydroxy or an O-glucuronic acid substituent;
and wherein any nitrogen atom in the compound of formula 5 is optionally bound to an oxygen to form an N-oxide moiety (—N→O).

14. The compound of claim 11, wherein the compound is N-[3-[4-(4-Hydroxy-3-methyl-phenylamino)-quinazolin-6-yl]allyl]-2-methoxy-acetamide.

15. A method for the treatment of abnormal cell growth in a mammal comprising administering to said mammal an amount of a compound of claim 13 that is effective in treating abnormal cell growth.

16. A method according to claim 15, wherein said abnormal cell growth is cancer.

17. A method according to claim 16, wherein said cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

18. A method for the treatment of abnormal cell growth in a mammal which comprises administering to said mammal an amount of a compound of claim 13 that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

19. A pharmaceutical composition for the treatment of abnormal cell growth in a mammal comprising an amount of a compound of claim 13 that is effective in treating abnormal cell growth, and a pharmaceutically acceptable carrier.

20. A kit for the treatment of abnormal cell growth comprising a) a pharmaceutical composition comprising a compound of claim 13 and a pharmaceutically acceptable carrier, vehicle or diluent; and b) instructions describing a method of using the pharmaceutical composition for treating the abnormal cell growth.
